(19) United States
(12) Patent Application Publication
(10) Pub. No.: US 2020/0069829 A1
(43) Pub. Date: Mar. 5, 2020

(54) MACHINE FOR TREATING OBJECTS, IN PARTICULAR FOR WASHING, THERMAL DISINFECTION AND/OR STERILIZATION OF OBJECTS

(71) Applicant: STEELCO S.P.A., Riese Pio X (IT)

(72) Inventor: Ivone CAPOVILLA, Loria (IT)

(73) Assignee: SteelCo S,p.a., Riese Pio X (IT)

(21) Appl. No.: 16/348,328

(22) PCT Filed: Nov. 8, 2017
(86) PCT No.: PCT/EP2017/078597

§ 371 Date: May 8, 2019

(30) Foreign Application Priority Data
Nov. 8, 2016 (IT) 102016000112539

(51) Int. Cl. A61L 2/24; A61L 2/07; B08B 3/04

(52) U.S. Cl. CPC A61L 2/24; A61L 2/07; B08B 3/041; A61L 2202/24; A61L 2202/122; A61L 2202/14; A61L 2202/17

(57) ABSTRACT

Machine for treating objects, including at least one treatment chamber configured to house at least one object-carrying container; the machine has at least one movement apparatus to move the object-carrying container provided with two joining means which can be selectively associable with the object-carrying container and configured to make said apparatus assume at least a first active position of insertion of the object-carrying container inside the chamber and at least a second active position of extraction of the object-carrying container from the chamber.

### FIELD OF THE INVENTION

The present invention concerns a machine for treating objects, in particular a so-called “tunnel” treatment machine, in which successive steps of treating objects can be carried out in one or more treatment chambers in which corresponding treatment zones are disposed one after the other.

The term treatment can include “in their entirety” both operations to pre-treat the objects, such as pre-washing, with hot or cold water and/or with detergents or other chemical products, as well as washing operations proper, and also drying operations. The term “treatment” also includes thermal disinfection, sterilization, generally in autoclaves, and decontamination by means of particularly aggressive and dangerous decontamination substances, detergents and/or chemical agents such as for example peracetic acid.

By way of example, the objects that can be treated in the treatment machine in question can be instruments used in the medical field, in the laboratory, for analysis or research, instruments used in the pharmaceutical field, or medical instruments, surgical instruments or similar or comparable instruments, but does not exclude the application of the present invention to the treatment of objects in general.

### BACKGROUND OF THE INVENTION

Machines are known for treating objects, generally contained in one or more object-carrying containers such as racks or suchlike which are fed inside at least one suitable treatment chamber by feed means, for example a belt, where the desired treatment cycle is performed.

Normally, at least one door is provided, associated with an entrance aperture, which is mobile and driven by an opening and closing mechanism to assume a high or open position in which it allows the objects to be treated to enter into the treatment chamber, and a low or closed position, in which, in cooperation with fluidic sealing means, it seals the washing chamber from the outside.

In single-door treatment machines, the entrance and exit of the containers and the objects treated that are contained therein are carried out through the same door. In pass-through treatment machines, two doors are provided, that is, one door associated with an entrance aperture, and one door associated with an exit aperture, normally in the opposite position to the entrance aperture, respectively for the entrance and exit of containers and the treated objects contained therein.

As we said, one or more object-carrying containers can be inserted inside the treatment chamber of the machine. When several object-carrying containers are inserted, they are positioned sequentially one after the other in the chamber.

In the case of a “single-door” treatment machine, a roller is provided, on which to place the object-carrying container or containers and situated in correspondence with the door of the machine. From that door, then, the containers with the objects to be treated enter and the containers with the treated objects exit.

In the case of a “pass-through” treatment machine, generally two rollers are provided, positioned on diametrically opposite sides of the machine, of which one side has an entrance door for containers with objects to be treated and the other side has an exit door for the containers with treated objects.

A first disadvantage of known treatment machines is the need to carry out at least the action of loading the object-carrying container or containers in a manual way, that is, by substantially pushing the container or containers with the objects to be treated on a roller, until the latter have completely entered the treatment chamber of the machine.

Another disadvantage of known treatment machines, moreover, especially for pass-through machines, is the need to provide, in correspondence with the roller located on the exit side of the container or containers containing treated objects, another slider to collect the container or containers. The slider is generally positioned transversely with respect to the roller that supports the container or containers with treated objects, so it is obvious that the bulk required on the exit side of the machine is rather high, especially if a battery of “pass-through” machines is provided in parallel.

It should also be underlined that in certain machines for treating objects, for example thermal disinfection machines and even more so in sterilization machines, the operating conditions inside the object treatment chamber are rather difficult, for example because of very high temperatures, for example up to 140° C., but also because of the working pressures and/or the substances used for the treatment. It is therefore a good rule that these sterilization machines should have, inside the treatment chamber, no motorized or automatic moving means of the object-carrying containers and/or sensors to detect their position. This constraint, therefore, involves considerable complications at least in the development of technical solutions for moving object-carrying containers from and to the treatment chambers.

Document WO-A-2015/145383 describes an apparatus to introduce and extract containers to/from a sterilization machine which provides attachment elements provided at one end of each of the racks and able to allow, if necessary, the reciprocal attachment of the racks.

Therefore, the apparatus does not describe an apparatus for moving object-carrying containers which provides attachment or joining means or elements that are activated alternately and allow the insertion and extraction of one or more containers to/from the treatment chamber.

Therefore, even the above apparatus, although it is advantageous if several containers located in sequence are used, to avoid, for example, jamming of the object-carrying containers, can have the disadvantages described above.

Document EP-A-1787730 concerns an apparatus which is provided with a rotating device to rotate the loading plane of a washing machine, in order to automatically discharge any residual water.

This document too does not describe a machine for treating objects provided with an effective movement apparatus for the containers inside it, provided in particular with attachment elements suitable to function alternately.

Other movement apparatuses which suffer from these disadvantages are also described in US-A-3595373, which describes the use of magnetic means for positioning pallets on a conveyor, EP-A-1787731, which concerns another known washing machine, and WO-A-2016/074066, which describes a known industrial washing system.

Other limitations and disadvantages of conventional solutions and technologies will be clear to a person of skill after reading the remaining part of the present description with reference to the drawings and the description of the embodiments that follow, although it is clear that the description of the state of the art connected to the present description must not be considered an admission that what is described here is already known from the state of the prior art.

There is therefore a need to obtain a machine for treating objects, in particular washing, thermal disinfection and/or sterilization of objects, which can overcome at least one of the disadvantages of the state of the art.

In particular, one purpose of the present invention is therefore to obtain a machine for treating objects provided with means to move one or more object-carrying containers, which means, in both the steps of insertion and extraction to/from the machine, eliminate or at least substantially limit manual operations.

It is another purpose of the present invention to obtain a treatment machine which has advantageously reduced bulk and which requires limited spaces, compared with known machines, in zones adjacent to the loading and/or unloading door or doors of one or more object-carrying containers.

Another purpose of the present invention is to obtain a treatment machine, in particular a pass-through machine, which is provided with means to insert and extract one or more containers which allow it to load the one or more containers with the objects to be treated on one side of the machine and which allow to recover said one or more empty containers from the same side of the machine, once the treated objects have been unloaded on the opposite side of the machine, from which said one or more containers temporarily exit in order to pick up the treated objects.

Another purpose of the present invention is to obtain an effective and automatic apparatus to move one or more object-carrying containers that allows to insert and extract the one or more object-carrying containers to/from any machine for treating objects, which can be driven alternately for the insertion or extraction of said one or more containers.

Another purpose of the present invention is to perfect a fast, effective and safe method to move one or more object-carrying containers, both in the insertion step and in the extraction of said one or more containers from a machine for treating objects.

The Applicant has devised, tested and embodied the present invention to overcome the shortcomings of the state of the art and to obtain these and other purposes and advantages.

### SUMMARY OF THE INVENTION

The present invention is set forth and characterized in the independent claims, while the dependent claims describe other characteristics of the invention or variants to the main inventive idea.

In accordance with the above purposes, a machine for treating objects, according to the present invention, in particular for washing, thermal disinfection and/or sterilization of objects, comprises at least one treatment chamber configured to house at least one object-carrying container.

According to one aspect of the invention, the treatment machine comprises at least one movement apparatus to move the object-carrying container provided with at least a first and a second attachment element which can be selectively activated, alternately one to the other, in order to attach to the object-carrying container and configured to make the movement apparatus assume at least a first active position of, insertion of the object-carrying container inside the treatment chamber and at least a second active position of extraction of the object-carrying container from the chamber; the attachment elements cooperate with a corresponding drive unit, configured to automatically alternate the active and inactive position, so that, when one of the attachment elements is driven in the active position, the other attachment element automatically moves into the inactive position, and vice versa.

If it is provided that the chamber houses a plurality of object-carrying containers located in sequence, the movement apparatus can be able to engage, in the first active position, with the first object-carrying container of the plurality of object-carrying containers and can be able to engage, in the second active position, with the last object-carrying container of the plurality of object-carrying containers.

The machine according to the invention can comprise a first access aperture to the treatment chamber located on a first side of the machine and a second access aperture to the treatment chamber located on a second side of the machine; a first movement apparatus can be positioned in proximity to the first access aperture and a second movement apparatus can be positioned in proximity to the second access aperture.

The invention also concerns a movement apparatus to move one or more object-carrying containers in a treatment machine, in particular a machine for washing, thermal disinfection and/or sterilization of objects, comprising at least two joining means which can be selectively activated, alternately one to the other, in order to be associable with the object-carrying container and configured to define at least a first active position of insertion of the object-carrying container inside a treatment chamber and at least a second active position of extraction of the object-carrying container from the chamber.

The invention also concerns a method to move one or more object-carrying containers in a treatment machine, in particular a machine for washing, thermal disinfection and/or sterilization of objects, the method comprising: the automated insertion of one or more object-carrying containers with objects to be treated inside a treatment chamber by means of a movement apparatus; the extraction of the movement apparatus from the chamber and the consequent treatment of the objects in the closed chamber; the opening of the chamber, when treatment is completed, and the new insertion of the movement apparatus inside the chamber; and the automated extraction of the one or more object-carrying containers with the objects which have been treated.

These and other aspects, characteristics and advantages of the present disclosure will be better understood with reference to the following description, drawings and attached claims. The drawings, which are integrated and form part of the present description, show some forms of embodiment of the present invention, and together with the description, are intended to describe the principles of the disclosure.

The various aspects and characteristics described in the present description can be applied individually where possible. These individual aspects, for example aspects and characteristics described in the attached dependent claims, can be the object of divisional applications.

It is understood that any aspect or characteristic that is discovered, during the patenting process, to be already known, shall not be claimed and shall be the object of a disclaimer.

### BRIEF DESCRIPTION OF THE DRAWINGS

These and other characteristics of the present invention will become apparent from the following description of some embodiments, given as a non-restrictive example with reference to the attached drawings wherein:

FIG. 1 is partial three-dimensional view of a treatment machine for objects, for example a “pass-through” machine, provided with a movement apparatus to move one of more object-carrying containers;

FIG. 2 is a plan view of the treatment machine in FIG. 1;

FIG. 3 is a plan view, partly in section, of the movement apparatus of the object-carrying containers;

FIG. 4 is a lateral elevation view, partly in section, of the movement apparatus in an extraction configuration of the object-carrying container or containers from the machine;

FIG. 5 is a lateral elevation view, partly in section, of the movement apparatus in an insertion configuration of the object-carrying container or containers into the machine;

FIGS. 6a-6d show some insertion and extraction steps of a pair of object-carrying containers into and from the treatment machine in FIG. 1 and FIG. 5.

To facilitate comprehension, the same reference numbers have been used, where possible, to identify identical common elements in the drawings. It is understood that elements and characteristics of one embodiment can conveniently be incorporated into other embodiments without further clarifications.

### DETAILED DESCRIPTION OF SOME EMBODIMENTS

We shall now refer in detail to the various embodiments of the present invention, of which one or more examples are shown in the attached drawings. Each example is supplied by way of illustration of the invention and shall not be understood as a limitation thereof. For example, the characteristics shown or described insomuch as they are part of one embodiment can be adopted on, or in association with, other embodiments to produce another embodiment. It is understood that the present invention shall include all such modifications and variants.

Before describing these embodiments, we must also clarify that the present description is not limited in its application to details of the construction and disposition of the components as described in the following description using the attached drawings. The present description can provide other embodiments and can be obtained or executed in various other ways. We must also clarify that the phraseology and terminology used here is for the purposes of description only, and cannot be considered as limitative.

With reference to the attached drawings, a treatment machine 10 comprises at least one treatment chamber 11 in which one or more object-carrying containers, for example the object-carrying containers 12 and 13, can be positioned.

The object-carrying containers 12 and 13 can be in the form of racks or suchlike.

Typically, the object-carrying containers 12 and 13 are provided, substantially at the base, with a series of bars 14.

The bars 14 are suitably located at an adequate reciprocal distance, generally to facilitate the passage of liquids and/or fluids, and are directed in a substantially transverse direction with respect to a direction for inserting and extracting the object-carrying containers 12 and 13 into and out of the chamber 11. The bars 14 can also be provided in cross-configuration, although portions of the bars 14 are provided that are transverse as specified above.

Alternately, it would be possible to provide only one object-carrying container, or more than two object-carrying containers located in sequence.

The fact that two or more object-carrying containers 12 and 13 located in sequence are provided is useful, for example, if the user has problems of bulk for the operations of inserting and extracting the object-carrying containers from the treatment machine 10, therefore providing a single object-carrying container could cause movement problems.

The length of the object-carrying container or the object-carrying containers 12 and 13 located in sequence is normally such that substantially the whole length of the treatment chamber 11 is occupied.

Hereafter in the description, reference will be made, by way of non-restrictive example, to the example shown of two object-carrying containers 12 and 13 located in sequence, and a pass-through 10 treatment machine. As will be easily appreciated, however, the present invention can also refer to a single-door treatment machine and/or the use of a single object-carrying container or the use of more than two object-carrying containers.

The treatment chamber 11 will be provided inside with means, not shown in the drawings, useful for performing a specific treatment of the objects put in the object-carrying containers 12 and 13, for example washing, thermal disinfection, sterilization means or other.

The chamber 11 of the treatment machine 10, in this case by way of example a “pass-through” machine, comprises an aperture 15 for inserting the object-carrying containers 12, 13 with the objects to be treated and an aperture 16 for extracting the object-carrying containers 12, 13 with the objects treated.

The apertures 15 and 16 are positioned on opposite sides of the chamber 11 and are opened or closed by means of corresponding doors, not shown in the drawings, which can be driven manually or automatically.

The opening and closing doors 15 and 16 are normally sliding doors, for example shutter doors or suchlike, and are able to ensure adequate sealing once the chamber 11 has been closed to treat objects located in the object-carrying containers 12, 13.

Inside the treatment chamber 11, the object-carrying containers 12, 13 rest on a pair of supports 17, for example a pair of parallel tracks.

The supports 17 are suitably raised with respect to the base 18 of the treatment chamber 11 and between them and the base 18 a compartment 46 is made, to temporarily house at least part of a mobile support 19, 19′ of a movement apparatus 20, 20′ to move the object-carrying containers 12 and 13.

The movement apparatus 20, 20′ is configured to assume a first active position in which it inserts the object-carrying containers 12, 13 inside the chamber 11, and at least a second active position in which it extracts the object-carrying containers 12, 13 from the chamber 11.

The first and second active positions of the apparatus 20, 20′ are obtained by means of suitable joining means 28, 29 which can be selectively activated, alternately with respect to each other, in order to be associable on each occasion, depending on the operation of loading, unloading or transfer/return to be performed, to the object-carrying container or object-carrying containers 12, 13.

In possible implementations, there is a plurality of joining means 28, 29, that is, two or more, for each apparatus 20, 20′.

In possible implementations, the joining means 28, 29 of each apparatus 20, 20′ include at least a first attachment element 28 and a second attachment element 29. In specific implementations, each apparatus 20, 20′ comprises only two joining means 28, 29, that is, only a first attachment element 28 and a second attachment element 29.

In some embodiments, it is possible to provide that the first attachment element 28 is distanced from the second attachment element 29 at least by the length of an object-carrying container 12 or 13.

In the case shown by way of example, the first attachment element 28 is distanced from the second attachment element 29 for a segment equal to the length of two object-carrying containers 12, 13.

In some embodiments, which can be combined with all the embodiments and implementations described here, for each apparatus 20, 20′ the first active position of the apparatus 20, 20′ is obtained, as we will see, by activating a first attachment element 28 and by deactivating a second attachment element 29.

In some embodiments, which can be combined with all the embodiments and implementations described here, for each apparatus 20, 20′ the second active position of the apparatus 20, 20′ is obtained by activating the second attachment element 29 and deactivating the first attachment element 28.

In particular, the first active position includes an attachment position of one of said attachment elements 28, 29 to the object-carrying container 12, 13, and the second inactive position comprises a release position of the other of the attachment elements 29, 28 from the object-carrying container 12, 13.

It is understood that with the term attachment elements 28 and 29 we mean both mechanical joining means, for example attachment elements of the hook or anchor type, like those shown in the drawings, and also possibly magnetic joining means, able to be activated for the insertion and extraction of the object-carrying container, or a different kind of mechanical, electromechanical or magnetic or other means or elements.

In possible implementations, the attachment elements 28 and 29 can be activated in the sense that they are mobile to be selectively, and alternately, associable with the specific object-carrying container 12, 13.

Hereafter in the description, by way of non-restrictive example, reference will be made to the attachment elements 28 and 29 shown as selectively activated joining means.

The mobile support 19, 19′ substantially comprises a box-like body that develops in the direction of insertion and extraction of the object-carrying containers 12 and 13 to/from the treatment chamber 11, and thus in a substantially longitudinal direction.

Inside the mobile support 19, 19′ a shaft 21, 21′ is housed, on which at least one pair of cams 22 and 23 are positioned.

The shaft 21, 21′ can be rotated in one direction or the other by an actuator 34 associated with one end 35 of the shaft 21, 21′ that emerges from the mobile support 19, 19′.

The actuator 34 is associated with the end 35 of the shaft 21, 21′ by means of suitable motion transmission means 36, 36′.

The actuator 34, by lifting and lowering the corresponding rod 37, can allow a certain rotation of the shaft 21, 21′ in one direction or the other. Such rotation can be, for example, about 90°.

The actuator 34, the shaft 21, 21′ and the cams 22, 23 thus represent a drive unit of the attachment elements 28 and 29, configured to automatically change the active and inactive position, so that when one of the attachment elements 28, 29 is driven in the active position, the other attachment element 29, 28 automatically moves to the inactive position, and vice versa.

The drive unit 34, 21, 21′, 22, 23, which essentially allows to convert or transform the rotational motion of the shaft 21, 21′ into a lifting or lowering motion of the attachment elements 28, 29 could also be made in a different way to that shown, therefore it could provide actuation means, in particular mechanical motion conversion or transformation means, different from the cams 22 and 23 cooperating with the corresponding attachment elements 28 and 29.

The drive unit could therefore also be selected, for example, from a group comprising: screw actuators such as a screw jack, ball screw actuators and roller screw actuators, or a wheel and axle, for example a drum, gear, pulley or shaft, actuators such as a lifting cable, a winch, a rack and a pinion group, a chain transmission, a belt or strap transmission, rigid chain actuators and rigid belt or strap actuators, or others.

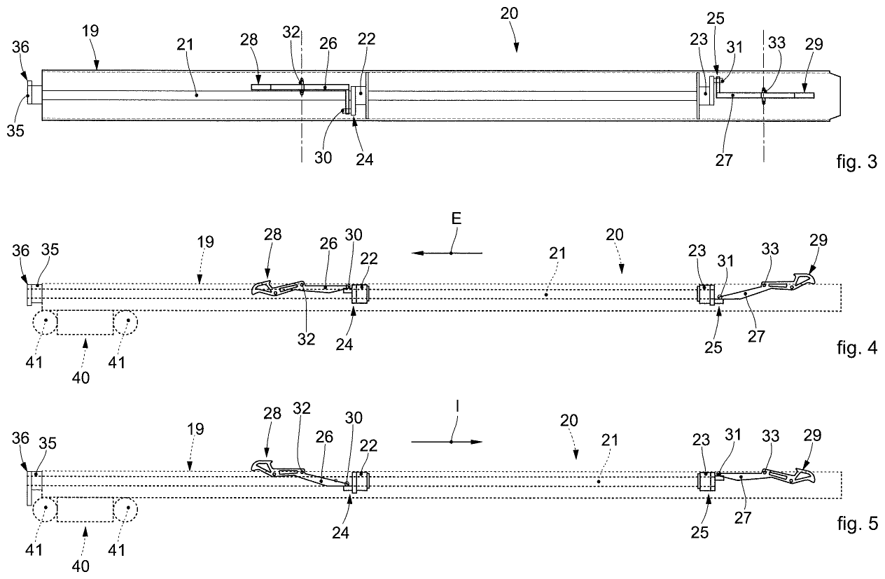

Therefore, returning to the description, as shown by way of non-restrictive example, each of the cams 22 and 23 of the shaft 21, 21′ is connected, by means of a corresponding lever mechanism 24 and 25, to an end 26 and 27 of an attachment element 28 and 29, see in particular FIG. 3, in which the box-like body of the mobile support 19 is shown without the upper part, so as to observe the inside.

The end 26 of the attachment element 28 is connected to the lever mechanism 24 by means of a rotation pin 30. The rotation pin 30 is raised and lowered along with the lever mechanism 24 and the end 26 of the attachment element 28.

The end 27 of the attachment element 29 is connected to the lever mechanism 25 by means of a rotation pin 31. The rotation pin 31 is raised and lowered along with the lever mechanism 25 and the end 27 of the attachment element 29.

Each of the attachment elements 28 and 29 is connected to a fixed rotation pin 32 and 33. The rotation pin 32, 33 is supported by the box-like body of the mobile support 19, 19′.

The attachment element 28 is able to allow an insertion movement in direction I of the object-carrying containers 12 and 13 inside the chamber 11, FIG. 5, while the attachment element 29 is able to allow an extraction movement in direction E of the object-carrying containers 12 and 13 from the treatment chamber 11, FIG. 4.

To this end, the attachment element 28 can have a concavity facing in direction I and the attachment element 29 can have a concavity facing in direction E.

The cams 22 and 23 and hence their corresponding lever mechanisms 24 and 25 are disposed in positions offset with respect to the shaft 21, 21′, for example in diametrically opposite positions, see in particular FIG. 3.

The attachment elements 28 and 29 protrude alternately from suitable slits 38 and 39 made on the surface of the mobile support 19, 19′.

The drive of the actuator 34, as will be seen below, automatically allows to lift and lower the attachment elements 28 and 29 alternately: therefore, by rotating the shaft 21, 21′ in a first direction, following a first travel of the actuator 34, there will be a lifting of the attachment element 29 into an active position and a lowering of the attachment element 28 into an inactive position; on the contrary, by rotating the shaft 21, 21′ in the other direction, following a second travel of the actuator 34, in the opposite direction to the first travel, there will be a lowering of the attachment element 29 into an inactive position and a lifting of the attachment element 28 into an active position.

As we said above, therefore, the rotational movement of the shaft 21, 21′ is converted into an alternate lifting or lowering movement of the attachment elements 28, 29.

When one of the attachment elements 28, 29 is raised and hence in an active position, it is able to engage with one of the bars 14 of the object-carrying containers 12, 13, while the other attachment element 29, 28 is lowered and in the inactive position, thus substantially housed retracted inside the mobile support 19, 19′ and thus in a non-interfering position with the object-carrying containers 12, 13.

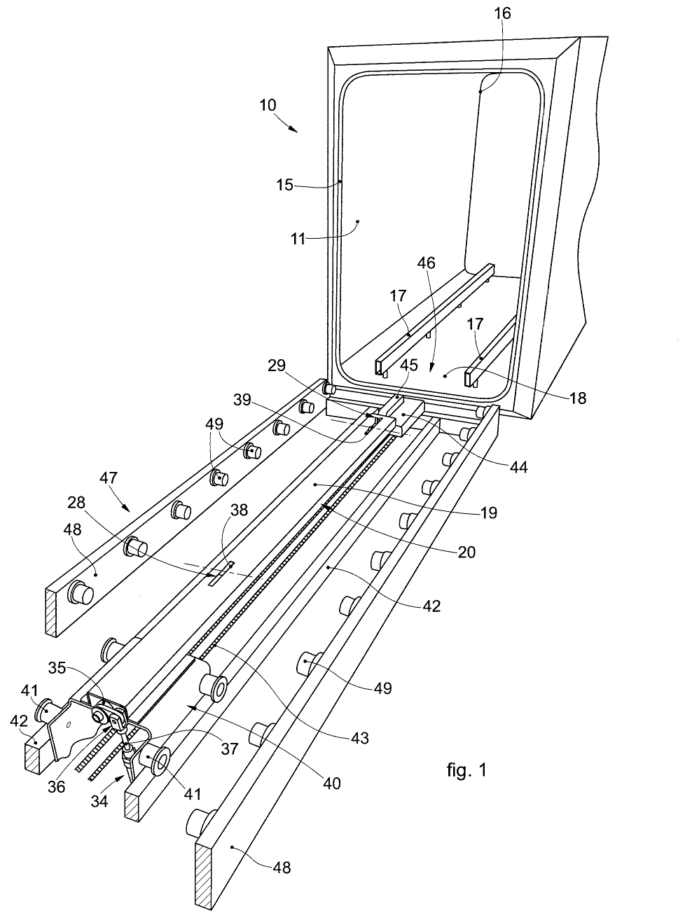

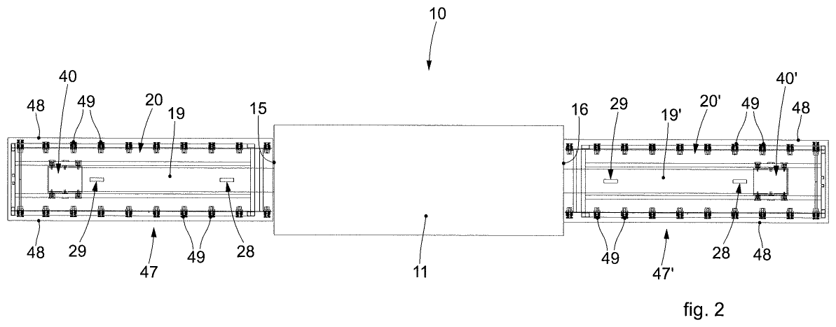

In FIG. 1, by way of example, it has been assumed that the attachment element 29 is raised and in the active position, while the attachment element 28 is lowered and in the inactive position.

The mobile support 19, 19′ of the movement apparatus 20, 20′ is housed on a slider 40, 40′.

On each side the slider 40, 40′ comprises rollers or wheels 41, resting on corresponding sliding guides 42.

The slider 40, 40′ can be translated along the sliding guides 42, in one direction or the other, and by any suitable drive mean, for example an electric motor or other.

The slider 40, 40′, in this example, is associated with a branch of a flexible motion transmission element 43, for example a chain, belt, or suchlike, wound at least around one pair of return wheels, one of which will be connected to said suitable drive mean.

The mobile support 19, 19′, near the aperture 15, 16 of the treatment chamber 11, can rest on a support 44.

On the support 44, a ramp 45 can be made, for inserting the mobile support 19, 19′ of the movement apparatus 20, 20′ into the compartment 46 made between the supports 17.

The apparatus 20, 20′ for moving the object-carrying containers 12, 13, can therefore be moved substantially bi-directionally along the guides 42, so that at least part of it, and in particular the mobile support 19, 19′, is housed in the compartment 46 and in such a way that the object-carrying containers 12, 13 are correctly positioned resting on the supports 17.

The object-carrying containers 12, 13 outside the treatment chamber 11 rest on corresponding rollers 47, 47′: a first roller 47, located in correspondence with the entrance aperture 15 of the object-carrying containers 12, 13 with the objects to be treated in the treatment room 11; and a second roller 47′, located in correspondence with the exit aperture 16 of the object-carrying containers 12, 13, with the objects treated in the treatment chamber 11.

Each roller 47, 47′ comprises at least one pair of guides 48 provided with rollers 49: the object-carrying containers 12, 13 are supported and can slide on said rollers 49.

The object-carrying containers 12, 13, for example racks, in which the objects to be treated are disposed, are positioned on the roller 47 which is located near the entrance aperture 15 of the treatment chamber 11. The door associated with the aperture 15 in this case will be open. On the contrary, it can be assumed that the door associated with the aperture 16 is closed.

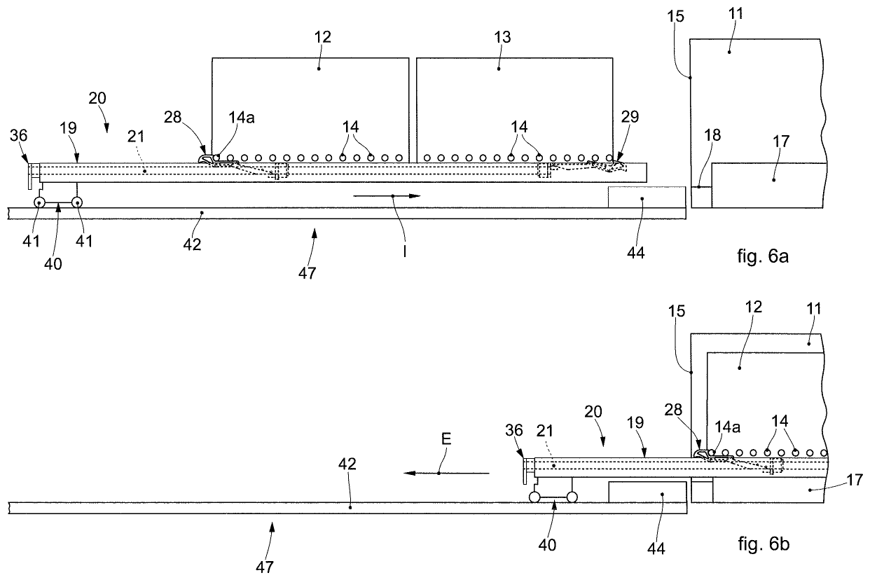

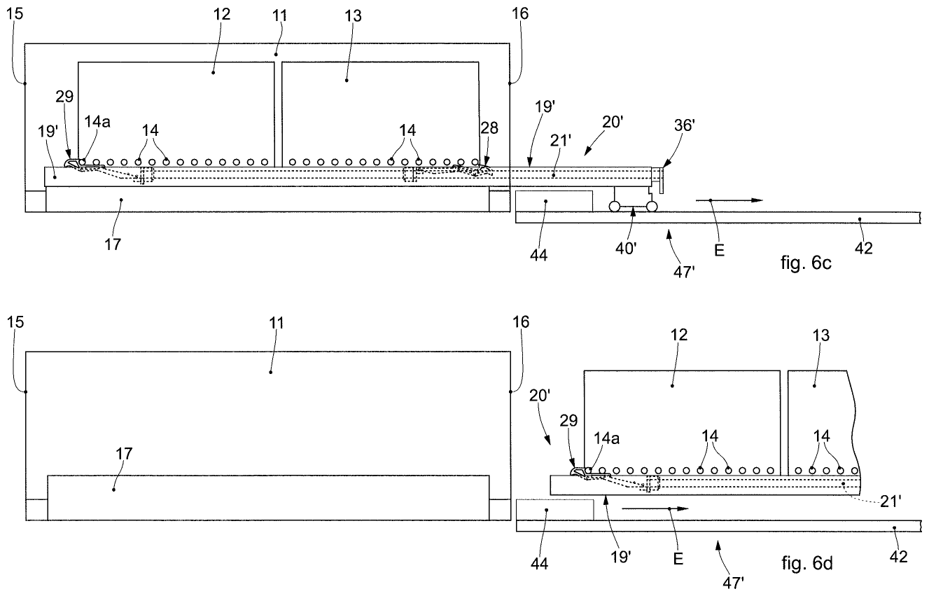

The object-carrying containers 12, 13 are positioned on the roller 47 in sequence and so that one of the transverse bars 14 of the first object-carrying container, that is, the object-carrying container 12, rests against the first attachment element 28, positioned in the raised position, see FIG. 6a. The bar that cooperates with the attachment element 28 can be, for example, the first bar 14a. The attachment element 29, on the other hand, is in a lowered position and therefore does not interfere with the object-carrying containers 12, 13.

Lifting the attachment element 28 and lowering the attachment element 29 has been carried out, preferably prior to positioning the object-carrying containers 12, 13 on the roller 47, by means of the actuator 34.

In particular, to pass for example from the configuration shown in FIG. 4 to that in FIG. 5, hence to activate the attachment element 28 and deactivate the attachment element 29, the actuator 34 is driven, for example, so as to recall the rod 37 and to cause a certain lowering of the transmission elements 36; this lowering of the transmission elements 36 causes a rotation of the shaft 21, with a consequent rotation of the cam 22 by a certain angle and a lowering of the lever mechanism 24. The lowering of the lever mechanism 24 determines the rotation of the attachment element 28 around the fixed pin 32, so as to rise and become active, as in FIG. 5. Also, the cam 23 associated with the attachment element 29 rotates by a certain angle, but being suitably offset with respect to the cam 22, the rotation of the shaft 21 will result in a lifting of the lever mechanism 25. The lifting of the lever mechanism 25 determines the rotation of the attachment element 29 around the fixed pin 33, so as to lower and become inactive, as in FIG. 5.

The actuator 34, as we said, can cause the shaft 21 to rotate in the opposite direction to the one described above, so that the actuator 34, for example, by removing the rod 37, could again cause the lifting and the active configuration of the attachment element 29 and the consequent lowering and the inactive configuration of the attachment element 28, so as to pass from the configuration shown in FIG. 5 to the configuration in FIG. 4.

Let us therefore suppose that we are in the situation of FIG. 6a, with the bar 14a of the object-carrying container 12 engaged with the attachment element 28 in the active position.

The slider 40 is driven in direction I so that it slides along the guides 42 and so that the mobile support 19 is at least partly inserted between the two supports 17, that is, in the compartment 46 of FIG. 1. The attachment element 28 is substantially configured to thrust and move the first object-carrying container 12 inside the chamber 11 through the aperture 15.

The object-carrying container 12 slides along the roller 47 and pushes the object-carrying container 13, which also slides on the roller 47.

The slider 40 of the drive apparatus 20 is stopped when, substantially, the attachment element 28 is located in correspondence with the entrance aperture 15 of the chamber 11, or has just gone past it, see FIG. 6b.

In this situation, both the object-carrying containers 12 and 13 are correctly positioned inside the chamber 11 and resting on the corresponding supports 17.

The mobile support 19 of the movement apparatus 20 can then be extracted from the chamber 11: the slider 40 is in this case translated in direction E, FIG. 6b, so that it comes out completely from the chamber 11 and it is thus possible to close the door of the aperture 15.

In the step of extracting the mobile support 19 and hence the movement apparatus 20, the attachment element 28 is kept in the active position, as it does not interfere with the bars 14 and 14a of the object-carrying container 12. The other attachment element 29 is therefore kept in the inactive position.

Once the mobile support 19 of the movement apparatus 20 has been completely removed and the door of the aperture 15 has been closed, the treatment of the objects contained in the object-carrying containers 12 and 13 is performed inside the chamber 11. Of course, the door of the aperture 16 will also be closed.

At the end of the treatment of the objects inside the chamber 11, the door of the exit aperture 16 of the object-carrying containers 12, 13 is opened, with the objects treated.

The movement apparatus 20′ is driven so that the slider 40′ takes the mobile support 19′ inside the chamber 11. In this step, the attachment element 29 of the apparatus 20′ will be in the inactive position, therefore lowered, while the attachment element 28 of the apparatus 20′ will be in the active position, therefore raised.

When the mobile support 19′ has been properly inserted into the compartment made between the supports 17 of the chamber 11 through the aperture 16, the slider 40′ is stopped and then the actuator associated with the transmission elements 36 of the apparatus 20′ is driven. The actuator can be totally similar to the actuator 34 shown in FIG. 1 and associated with the apparatus 20.

By means of this actuator, the shaft 21′ disposed inside the mobile support 19′, FIG. 6c, is rotated so that the attachment element 29 assumes an active position and the attachment element 28 assumes an inactive position.

The attachment element 29 of the apparatus 20′ thus cooperates with one of the transverse bars 14 of the object-carrying container 12, for example the bar 14a: by driving the slider 40′ in direction E, the movement apparatus 20′ extracts the object-carrying containers 12 and 13 from the chamber 11. This extraction takes place thanks to the attachment element 29 of the apparatus 20′ which engages with the object-carrying container 12 which in turn thrusts the object-carrying container 13 out of the chamber 11.

When extraction is completed, see FIG. 6d, the object-carrying containers 12 and 13 with the treated objects are both on the roller 47′.

Once the object-carrying containers 12 and 13 have been emptied of the treated objects, it is possible, thanks to the apparatus 20, 20′, to return the object-carrying containers 12, 13, this time empty, to the roller 47, from where they started with the objects to be treated.

The treatment chamber 11 in this step can thus be used as a container-passing apparatus to return the object-carrying containers 12, 13 to the side from which they started.

This aspect can be extremely advantageous when, in the proximity of the exit roller 47′ of the object-carrying containers 12, 13 there is a limited space and in any case not suitable to maneuver the object-carrying containers 12, 13.

To allow the return of the object-carrying containers 12, 13 to the roller 47 from which they started, as can easily be understood, it is necessary to operate in the opposite way to what has just been described.

In particular, assuming that the door of the aperture 16 is still open after the extraction shown in FIG. 6d, it can be assumed, as we said, that the object-carrying containers have been emptied of the treated objects.

At this point, by activating the attachment element 28 of the apparatus 20′ and deactivating the attachment element 29 of the apparatus 20′, it is possible to push the empty object-carrying containers 12, 13 inside the chamber 11.

After the insertion of the empty object-carrying containers 12, 13 inside the chamber 11 is completed, the mobile support 19′ of the movement apparatus 20′ is extracted from the aperture 16.

It should be noted that, preferably, when the empty object-carrying containers 12, 13 have been inserted inside the chamber 11, it is best to first close the door associated with the aperture 16 and then to open the door associated with the aperture 15, so as to prevent any possible contamination of the atmosphere of the “clean” side of the treatment machine 10 by the “dirty” side of the treatment machine 10, or respectively, the side where the roller 47′ facing toward the aperture 16 and the side where the roller 47 is positioned facing the aperture 15.

In general, therefore, when the door associated with the aperture 16 is open, the door associated with the aperture 15 should remain closed, or vice versa, in order to prevent the “clean” side of the treatment machine 10 from entering into communication in any way with the “dirty” side of the treatment machine.

Once the empty object-carrying containers 12 and 13 have been inserted into the chamber 11, the door associated with the aperture 16 is closed.

At this point, the movement apparatus 20 located on the roller 47 near the aperture 15 again intervenes, the door of which can be opened because the door of the aperture 16 has been closed.

The movement apparatus 20 has the attachment element 28 active and the attachment element 29 deactivated, so the mobile support 19 is inserted under the empty object-carrying containers 12, 13 so that the attachment element 29, in the inactive position, moves under one of the bars 14 of the empty object-carrying container 13.

At this point, thanks to the actuator 34 and as explained above, the attachment element 29 of the movement apparatus 20 is taken to the active position while the attachment element 28 is taken to the inactive position, passing substantially from the situation shown in FIG. 5 to the situation in FIG. 4.

Thanks to the subsequent drive of the slider 40 in direction E, it is possible to extract the two empty object-carrying containers 12, 13 and position them correctly on the roller 47.

Thanks to the movement apparatus 20, 20′ with a double attachment element 28 and 29, it is therefore possible to introduce object-carrying containers 12, 13 with objects to be treated on one side of a pass-through treatment machine 10 and extract them, once emptied, from the same side from which they were introduced, for example the side of the aperture 15 with its corresponding roller 47. The treated objects will instead be suitably removed from the opposite side of the machine, in this example the side of the aperture 16 with corresponding roller 47′.

It is obvious that the present movement apparatus 20, 20′ can advantageously be used also for “single-door” type machines, that is, machines by means of which the object-carrying containers are loaded with objects to be treated on the same side as the unloading of the object-carrying containers with treated objects.

Assuming that a “single-door” treatment machine is being used, provided for example only with the aperture 15, it is easily understood that the apparatus 20 would allow, in an automated manner, both the insertion and the extraction of one or more object-carrying containers 12, 13 into/from the treatment chamber 11.

In the insertion step of the object-carrying containers 12, 13 into the chamber 11, the attachment element 28 is activated and the attachment element 29 is deactivated, see FIG. 5, so that the mobile support 19 introduces the object-carrying containers 12, 13 into the chamber 11. The mobile support 19 would then be extracted and the door associated with the aperture 15 would be closed for treatment.

Upon completion of the treatment, the door associated with the aperture 15 would be opened again and the mobile support 19 would be introduced again into the compartment 46 under the object-carrying containers 12, 13, with the attachment element 28 always in the active position and the attachment element 29 always in the inactive position.

When the insertion of the mobile support 19 is complete, and thanks to the actuator 34, the attachment element 29 would be taken to the active position and the attachment element 28 would be taken to the inactive position, FIG. 4, so as to engage the object-carrying container 13 and take both the object-carrying containers 12, 13 outside by extracting the device 19 from the chamber 11.

As we have seen, the extraction of the mobile support 19 is performed by driving the slider 40 in direction E, while the insertion of the mobile support is performed by driving the slider in direction I.

As previously mentioned and as easily understood, the movement apparatus 20, 20′ described could also be used in the case of a single object-carrying container of suitable length.

Furthermore, by varying the number and/or positioning of the attachment elements 28 and 29 on the removably connected mobile support 19, 19′, it would be possible to provide object-carrying containers of different lengths or object-carrying containers which do not substantially occupy the entire extension of the treatment chamber 11.

Since it is provided with a substantially mechanical movement apparatus 20, 20′ and is able to be inserted and disengaged from the chamber 11, the present treatment machine can also operate under difficult and complex operating conditions, for example those of a sterilization machine, where the working temperatures can be rather high, even up to 140° C., and therefore, where it is not advisable to use motorized means for moving the object-carrying containers inside the chamber.

It is clear that modifications and/or additions of parts can be made to the treatment machine, in particular for sterilization, as described heretofore, without departing from the field and scope of the present invention.

It is also clear that, although the present invention has been described with reference to some specific examples, a person of skill in the art shall certainly be able to achieve many other equivalent forms of treatment machine, in particular for sterilization, having the characteristics as set forth in the claims and hence all coming within the field of protection defined thereby.

In the following claims, the sole purpose of the references in brackets is to facilitate reading: they must not be considered as restrictive factors with regard to the field of protection claimed in the specific claims.

1. Machine for washing, thermal disinfection and/or sterilization of objects, comprising at least one treatment chamber configured to house at least one object-carrying container, wherein it comprises at least one movement apparatus to move the object-carrying container provided with at least a first and a second attachment element which can be selectively activated, alternately one to the other, in order to be attached to the object-carrying container and configured to make said apparatus assume at least a first active position of insertion of the object-carrying container inside the chamber and at least a second active position of extraction of the object-carrying container from the chamber, said attachment elements cooperating with a corresponding drive unit, configured to automatically alternate the active and inactive position, so that, when one of said attachment elements is driven in the active position, the other attachment element automatically moves into the inactive position, and vice versa.

2. Machine as in claim 1, wherein if said chamber houses a plurality of object-carrying containers located in sequence, said apparatus is able to engage, in said first active position, with the first object-carrying container of said plurality of object-carrying containers and is able to engage, in said second active position, with the last object-carrying container of said plurality of object-carrying containers.

3. Machine as in claim 1, wherein said first and second attachment elements comprise at least a first active position of engagement with the object-carrying container and at least a second inactive position of disengagement and non-interference with the object-carrying container.

4. Machine as in claim 1, wherein said attachment elements cooperate with a shaft housed in the movement apparatus and provided with at least a first cam configured to activate/deactivate said first attachment element and with at least a second cam configured to deactivate/activate said second attachment element, or vice versa, said shaft being made to rotate in one direction or the other by means of a corresponding actuator.

5. Machine as in claim 1, further comprising a first access aperture to the treatment chamber located on a first side of the machine and a second access aperture to the treatment chamber located on a second side of the machine, a first movement apparatus being positioned in proximity to said first access aperture and a second movement apparatus being positioned in proximity to said second access aperture.

6. Machine as in claim 5, wherein said apertures are positioned on opposite sides of the treatment chamber, said first movement apparatus, disposed in proximity to said first aperture, being configured to insert into the chamber one or more object-carrying containers with objects to be treated, and to extract from the chamber said one or more object-carrying containers which have been emptied, and said second movement apparatus, disposed in proximity to said second aperture, being configured to extract from the chamber one or more object-carrying containers with objects which have been treated, and to insert into said chamber said one or more object-carrying containers which have been emptied.

7. Movement apparatus to move one or more object-carrying containers in a machine for washing, thermal disinfection and/or sterilization of objects as in claim 1, further comprising at least a first and a second attachment element which can be selectively activated, alternately one to the other, in order to attach to the object-carrying container and configured to define at least a first active position of insertion of the object-carrying container inside a treatment chamber and at least a second active position of extraction of the object-carrying container from the chamber, said attachment elements cooperating with a corresponding drive unit, configured to automatically alternate the active and inactive position, so that, when one of said attachment elements is driven in the active position, the other attachment element automatically moves into the inactive position, and vice versa.

8. Method to move one or more object-carrying containers in a machine for washing, thermal disinfection and/or sterilization of objects as in claim 1-and using a movement apparatus comprising, at least a first and a second attachment element which can be selectively activated, alternately one to the other, in order to attach to the object-carrying container and configured to define at least a first active position of insertion of the object-carrying container inside a treatment chamber and at least a second active position of extraction of the object-carrying container from the chamber, said attachment elements cooperating with a corresponding drive unit, configured to automatically alternate the active and inactive position, so that, when one of said attachment elements is driven in the active position, the other attachment element automatically moves into the inactive position, and vice versa, wherein said method comprises: the automated insertion of one or more object-carrying containers with objects to be treated inside a treatment chamber by means of said movement apparatus; the extraction of the movement apparatus from the chamber and the consequent treatment of the objects in the closed chamber; the opening of the chamber, when treatment is completed, and the new insertion of the movement apparatus inside the chamber; and the automated extraction of said one or more object-carrying containers with the objects which have been treated.

9. Method as in claim 8, wherein said step of inserting one or more object-carrying containers occurs from a first side of the chamber, by means of a first movement apparatus, and the step of extracting said one or more object-carrying containers with objects that have been treated occurs from a second side of the chamber, by means of a second movement apparatus.

10. Method as in claim 9, further comprising another step of inserting into the chamber said one or more object-carrying containers which have been emptied from said second side of the chamber by means of said second movement apparatus and a step of extracting from the chamber said one or more object-carrying containers which have been emptied from the first side of the chamber by means of said first movement apparatus.
